(19) United States
(12) Patent Application Publication
(10) Pub. No.: US 2024/0387046 A1
(43) Pub. Date: Nov. 21, 2024

(54) METHOD FOR TUMOR FRACTION ESTIMATION

(71) Applicant: Sophia Genetics S.A., Rolle (CH)

(72) Inventors: CHRISTIAN POZZORINI, Lausanne (CH); JONATHAN BIELER, Renens (CH); ZHENYU XU, Nyon (CH)

(73) Assignee: Sophia Genetics S.A., Rolle (CH)

(21) Appl. No.: 18/669,474

(22) Filed: May 20, 2024

Related U.S. Application Data
(60) Provisional application No. 63/467,853, filed on May 19, 2023.

(51) Int. Cl. G16H 50/20; G16B 20/20; G16B 30/10

(52) U.S. Cl. CPC G16H 50/20; G16B 20/20; G16B 30/10

(57) ABSTRACT

Provided may be a computer-implemented method for estimating a tumor fraction in a patient sample, comprising the steps of obtaining a catalog of tumor specific variants and whole genome sequencing data from the patient sample. Further, the method may comprise aligning reads to a reference genome; determining a measure of the signal supporting the presence, in the patient sample read alignment file, of variants in the catalog of tumor specific variants; and determining a measure of the noise associated with variants similar to variants in the catalog of tumor specific variants in the patient sample read alignment file. The method may comprise estimating, over iterations, k, the fraction of tumor (eTF) DNA in the patient sample given the measure of the signal and the measure of the noise at all tumor specific positions; and generating a final eTF and a list of somatic variants in the patient sample.

### CROSS-REFERENCE TO RELATED APPLICATIONS

The present application claims the benefit of U.S. Patent Application No. 63/467,853 for TUMOR FRACTION FROM CTDNA WHOLE GENOME SEQUENCE, filed May 19, 2023, the entire contents of which are incorporated herein by reference in their entirety.

### FIELD OF THE INVENTION

Methods described herein relate to genomic analysis in general, and more specifically to estimation of the tumor fraction in cfDNA based on variant calling data obtained from whole genome sequencing data from tumor-normal pairs and cfDNA.

### INTRODUCTION

Minimal residual disease (MRD) refers to the presence of a small number of cancer cells that remain in the body after treatment. Detection of MRD in cell-free DNA (cfDNA) found in the bloodstream requires sensitive laboratory methods to support detection of somatic variants that would result from cancer cell death, also known as circulating tumor DNA (ctDNA). Circulating DNA fragments are mainly short molecules with an average length of mononucleosome size that tends to be more fragmented in internucleosomal linkers and open chromatin regions. The challenges of detecting a small number of somatic variants in a limited number of available ctDNA molecules has been extensively studied (i.e., https://www.science.org/doi/10.1126/sciadv.abc4308).

One way of detecting the presence of ctDNA in cfDNA is to leverage the cumulative signal afforded by screening thousands of preidentified tumor-specific variants. These tumor-specific variants can be identified at diagnosis using approaches that allow profiling of a large fraction of the genome, typically whole genome sequencing or whole exome sequencing. Approaches to identify tumor-specific variants can rely on profiling of tumor and normal samples. Alternatively, tumor-only based approaches followed by filtering of likely germline variants have been known in the public domain.

After the identification of tumor-specific variants, cancer progression can be monitored by testing for their presence in cfDNA. The presence of these tumor-specific variants is evidence for the presence of ctDNA, minimal residual disease, and relapse. This analysis can be done by targeted analysis at high depth regions of the genome, where patient-specific variants are known to be found. However, given the restricted number of DNA copies of each site in cfDNA, this approach is not effective even when ultra-deep sequencing with error suppression is used to detect ctDNA when tumor fraction is lower than 0.1-1% (PMID: 29968853).

Alternatively, whole genome sequencing can be used to leverage information from a large number of tumor-specific variants to quantify the fraction of tumor DNA, also referred to herein as tumor fraction (TF), in cfDNA, and determine MRD. The focus of this application is SNVs and indels, but in principle a similar approach to the one described here may be applied to CNVs and other variants

To reduce costs of implementation associated with sequencing large genomes at high depth, some of the available methods are designed to allow the use of low coverage sequencing data. The use of low coverage sequencing data comes with the challenge of distinguishing the signal coming from ctDNA and from noise coming from data generation and sequencing workflows (e.g., PCR and sequencing errors). Several approaches can be used to reduce the impact of noise on tumor fraction estimation.

One approach (e.g., MRDetect, described in WIPO Pub. Nos. WO2019169044A1 and WO2019169042A1 and the basis of Ci2 technology) is to measure the noise in a control sample (for example, either a normal/germline sample collected at diagnostic or a panel of normal samples) and to subtract the noise from the cfDNA measurement. This is, for example, what is implemented in prior art WO2019169044A1, WO2019169042A1, MRDetect (https://pubmed.ncbi.nlm.nih.gov/32483360/). The main disadvantage of the aforementioned prior methods is that they rely on the assumption that the noise in the control sample is the same as in the cfDNA sample, which likely will be violated in some cases (https://www.biorxiv.org/content/10.1101/2022.01.17.476508v1.full).

An alternative approach that relies on leveraging the noise from cfDNA and using artificial intelligence (AI) has recently been proposed (MRD-EDGE, https://www.biorxiv.org/content/10.1101/2022.01.17.476508v1.full). MRD-EDGE is based on a machine learning method that was trained to recognize true somatic variants in cfDNA based on cfDNA data from three (3) common cancer types with high mutational burden (colon, lung and melanoma). One of the limitations of this approach (MRD-EDGE) is that it requires availability of a large, pre-filtered sets of somatic variants in cfDNA for training. In addition, the method was only tested in Colorectal, Melanoma and Lung cancer patients and, given the well-established tissue specific cancer signatures (https://www.nature.com/articles/s41586-020-1943-3), it is unclear whether this approach is applicable to other tumor types. Methods that are tumor agnostic, such as the method described in the description below, and don't rely on a large dataset of curated variants would be valuable.

In contrast with previous methods, the method herein aims to distinguish true variants from sequencing noise using information solely present in the sequencing data from the patient. The proposed method is based on estimating the tumor fraction in cfDNA based only on variant calling data obtained from whole genome sequencing data from tumor-normal pairs and cfDNA.

### SUMMARY

This Summary is provided to introduce a selection of concepts in a simplified form that are further described below in the Detailed Description. This Summary is not intended to identify key features or essential features, nor is it intended to limit the scope of the claims included herewith.

Provided may be a computer-implemented method for estimating a tumor fraction in a patient sample, comprising the steps of obtaining a catalog of tumor specific variants, wherein the catalog of tumor specific variants was assembled based on analysis of at least one patient sample at a baseline (t=0); and obtaining whole genome sequencing data from the patient sample. Further, the computer-implemented method may comprise the steps of aligning reads of the whole genome sequencing data from the patient sample to a reference genome to obtain a patient sample read alignment file; determining a measure of the signal supporting the presence, in the patient sample read alignment file, of variants in the catalog of tumor specific variants; and determining a measure of the noise associated with variants similar to variants in the catalog of tumor specific variants in the patient sample read alignment file. In a further embodiment, the computer-implemented method may comprise the steps of estimating, over one or more iterations, k, the fraction of tumor (eTF) DNA in the patient sample given the measure of the signal and the measure of the noise at all tumor specific positions; and generating a final eTF at a last iteration of the one or more iterations and a list of somatic variants in the patient sample.

In an embodiment, the step of estimating the eTF further comprising the steps of assigning a probability of being somatic to each variant in the catalog of tumor specific variants; computing a signal supporting the presence of the variants in the catalog of tumor specific variants in the patient sample using a first weighted sum; and computing a noise supporting the presence of the variants in the catalog of tumor specific variants in the patient sample using a second weighted sum. In such an embodiment, at k=0, the probability of being somatic of each variant in the catalog of tumor specific variants is 1, Psomatic^{k}[x]=1, wherein x is a tumor variant in the catalog of tumor specific variants and k is the number of a current iteration. In another embodiment, the probability of being somatic to each variant in the catalog of tumor specific variants, Psomatic^{k}[x], at k=0, is based on information for the variant obtained from a common variant database.

In one aspect of the computer-implemented method, the step of computing a probability of each tumor variant in the catalog of tumor specific variants being somatic, Psomatic^{k}[x], further comprising the steps of: computing, at k>0, for each variant in the catalog of tumor specific variants, the likelihood of the variant being germline (Lgermline[x]), wherein Lgermline[x] is Binomial(Ntot_signal[x], 1; Nalt_signal[x])+Binomial(Ntot_signal[x], 0.5; Nalt_signal[x]); computing, at k>0, for each variant in the catalog of tumor specific variants, a likelihood of the variant being somatic (Lsomatic^{k} [x]), wherein Lsomatic^{k} [x]=Binomial(Ntot_signal[x], TF^{k}; Nalt_signal[x]); and computing, at k>0, the probability of variant x being somatic, wherein Psomatic^{k}[x]=Lsomatic^{k} [x]/(Lsomatic^{k} [x]+Lgermline[x]).

In yet a further aspect of the computer-implemented method, the step of determining a fraction of reads in the read alignment file supporting presence of representative variants further comprising the steps of obtaining a list of representative genomic intervals; considering positions in each of the representative genomic intervals in the list of representative genomic intervals that have a same reference nucleotide base (ref nucleotide base) as variants in the catalog of tumor specific variants; excluding, from the positions in each of the list of representative genomic intervals having the same reference nucleotide base as variants in the catalog of tumor specific variants, and excluding positions comprising a variant with an allele frequency greater than 5% and variants in the catalog of tumor specific variants.

In an embodiment, in the list of representative genomic intervals, each of the representative genomic intervals are genomically neighboring, defined as a window of 100 bp centered around variants in the catalog of tumor specific variants. In the list of representative genomic intervals, each of the representative genomic intervals may share one or more similar characteristics as the genomic interval where variants in the catalog of tumor specific variants are located. The similar characteristics may include similar sequence composition and chromatin status.

The measure of the signal supporting the presence of the variants in the catalog of tumor specific variants in the patient sample using a first weighted sum may be defined as Signal=Nalt_signal/Ntot_signal, wherein Nalt_signal=sum(Nalt_signal[x]*Psomatic^{k}[x]) over all variants, x, and wherein Ntot_signal=sum(Ntot_signal[x]*Psomatic^{k}[x]) over all variants, x.

In an embodiment, the computer-implemented method may further comprise the steps of for each variant in the catalog of tumor specific variants, computing a number of reads in the patient sample that supports an alt sequence (Nalt_signal[x]); and for each variant in the catalog of tumor specific variants, computing a number of reads in the patient sample that span the variant (Ntot_signal [x]).

In an embodiment, the measure of the noise supporting the presence of the variants in the catalog of tumor specific variants in the patient sample using a second weighted sum is defined as Noise=Nalt_noise/Ntot_noise, wherein Nalt_noise=sum(Nalt_noise [x]*Psomatic^{k}[x]) over all variants, x, wherein Ntot_noise=sum(Ntot_noise [x]*Psomatic^{k}[x]) over all variants, x.

The computer-implemented method may further comprise the steps of determining a fraction of reads in the patient sample read alignment file supporting representative variants; computing a number of reads in the patient samples that supports an alt sequence (Nalt_noise[x]) at representative variant; and computing a number of reads in the patient samples that span the variant (Ntot_noise [x]) at representative variant.

As a nonlimiting example, the whole genome sequencing data from the patient sample has a coverage less than 100x. As another nonlimiting example, the whole genome sequencing data from the patient sample has a coverage less than 10x. In yet another nonlimiting example, the whole genome sequencing data from the patient sample has a coverage less than 5x.

In one embodiment, the catalog of tumor specific variants is obtained from whole genome sequencing data from tumor-normal pairs.

The computer-implemented method may further comprise the steps of determining, via variant calling methods, variants present in the tumor sequencing data at the baseline and the germline sequencing data at the baseline; classifying the variants present in the tumor sequencing data at the baseline and the germline sequencing data at the baseline as likely germline or likely somatic; and comparing the likely germline variants and the likely somatic variants to construct the catalog of tumor specific variants.

The step of classifying the variants present in the tumor sequencing data at the baseline and the germline sequencing data at the baseline as likely germline or likely somatic may further comprise the step of defining variants in the germline sequencing data at the baseline as likely germline.

The step of classifying the variants present in the tumor sequencing data at the baseline and the germline sequencing data at the baseline as likely germline or likely somatic may further comprise the step of defining variants in the tumor sequencing data at the baseline as likely germline if said variants are present in the variants of the germline sequencing data at the baseline.

In an embodiment, the step of classifying the variants present in the tumor sequencing data at the baseline and the germline sequencing data at the baseline as likely germline or likely somatic further comprises the step of defining variants not included in the germline sequencing data at the baseline as likely somatic.

The patient samples may comprise cell-free DNA (cfDNA). The tumor DNA may be circulating tumor DNA (ctDNA).

The step of estimating, over one or more iterations, k, the fraction (eTF) of tumor DNA in the patient sample given the measure of the signal and the measure of the noise at all tumor specific positions may further comprise utilizing an expectation-maximization algorithm.

In an embodiment, a variant is defined as being somatic if Psomatic^{k}[x] is greater than a predefined value, p.

In an aspect of the present disclosure, the whole genome sequencing data from the patient sample is obtained at a later time (t=1).

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIG. 1 is an illustrative block diagram of a system based on a computer configured to execute one or more aspects of the functionality described herein.

FIG. 2 is an illustration of a computing machine configured to execute one or more aspects of the functionality described herein.

FIG. 3 illustrates a next generation sequencing system according to certain embodiments of the present disclosure.

FIG. 4 illustrates a workflow for estimation of tumor fraction.

FIG. 5 illustrates a workflow depicting determination of the fraction of reads in a read alignment file for cfDNA.

FIG. 6 illustrates a workflow depicting an iterative method to estimate the fraction of circulating tumor DNA in a cfDNA sample.

FIGS. 7-8 illustrate block diagrams for the estimation of tumor fraction in circulating tumor DNA.

### DETAILED DESCRIPTION

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

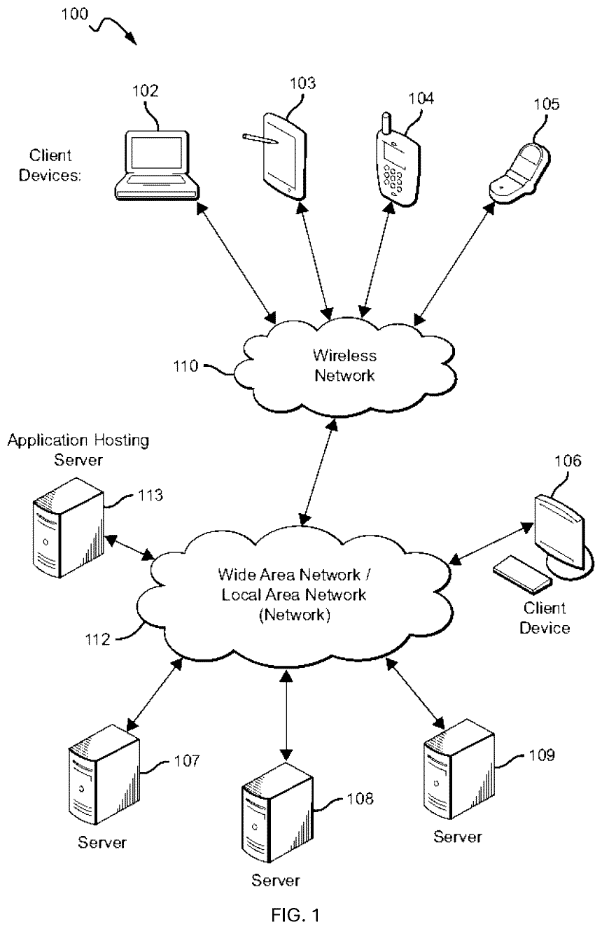

FIG. 1 illustrates components of one embodiment of an environment in which the invention may be practiced. Not all of the components may be required to practice the invention, and variations in the arrangement and type of the components may be made without departing from the spirit or scope of the invention. As shown, the system 100 includes one or more Local Area Networks (“LANs”)/Wide Area Networks (“WANs”) 112, one or more wireless networks 110, one or more wired or wireless client devices 106, mobile or other wireless client devices 102-105, servers 107-109, and may include or communicate with one or more data stores or databases. Various of the client devices 102-106 may include, for example, desktop computers, laptop computers, set top boxes, tablets, cell phones, smart phones, smart speakers, wearable devices (such as the Apple Watch) and the like. Servers 107-109 can include, for example, one or more application servers, content servers, search servers, and the like. FIG. 1 also illustrates application hosting server 113.

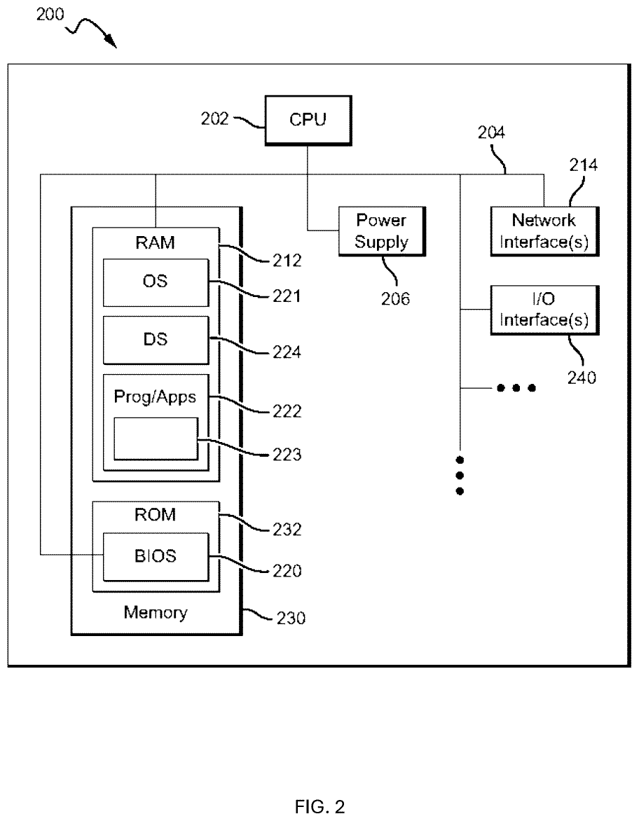

FIG. 2 illustrates a block diagram of an electronic device 200 that can implement one or more aspects of an apparatus, system and method for increasing mobile application user engagement (the “Engine”) according to one embodiment of the invention. Instances of the electronic device 200 may include servers, e.g., servers 107-109, and client devices, e.g., client devices 102-106. In general, the electronic device 200 can include a processor/CPU 202, memory 230, a power supply 206, and input/output (I/O) components/devices 240, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 200. A touchscreen may determine whether a user is providing input by, for example, determining whether the user is touching the touchscreen with a part of the user's body such as his or her fingers. The electronic device 200 can also include a communications bus 204 that connects the aforementioned elements of the electronic device 200. Network interfaces 214 can include a receiver and a transmitter (or transceiver), and one or more antennas for wireless communications.

The processor 202 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 230, which can include Random Access Memory (RAM) 212 and Read Only Memory (ROM) 232, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 221, data storage 224, which may include one or more databases, and programs and/or applications 222, which can include, for example, software aspects of the program 223. The ROM 232 can also include Basic Input/Output System (BIOS) 220 of the electronic device.

Software aspects of the program 223 are intended to broadly include or represent all programming, applications, algorithms, models, software and other tools necessary to implement or facilitate methods and systems according to embodiments of the invention. The elements may exist on a single computer or be distributed among multiple computers, servers, devices or entities.

The power supply 206 contains one or more power components and facilitates supply and management of power to the electronic device 200.

The input/output components, including Input/Output (I/O) interfaces 240, can include, for example, any interfaces for facilitating communication between any components of the electronic device 200, components of external devices (e.g., components of other devices of the network or system 100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 240 and the bus 204 can facilitate communication between components of the electronic device 200, and in an example can case processing performed by the processor 202.

Where the electronic device 200 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system and method of the Engine. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 102-106, as may be used in an example apparatus, system and method embodying the Engine, may range widely in terms of capabilities and features. For example, a cell phone, smart phone or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 102-106, for example, as may be used in an example apparatus, system and method implementing the Engine, may run a variety of operating systems, including personal computer operating systems such as Windows, iOS or Linux, and mobile operating systems such as iOS, Android, Windows Mobile, and the like. Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MMS, playing games, receiving advertising, watching locally stored or streamed video, or participating in social networks.

In example aspects of the apparatus, system and method implementing the Engine, one or more networks, such as networks 110 or 112, for example, may couple servers and client devices with other computing devices, including through wireless network to client devices. A network may be enabled to employ any form of computer readable media for communicating information from one electronic device to another. The computer readable media may be non-transitory. Thus, in various embodiments, a non-transitory computer readable medium may comprise instructions stored thereon that, when executed by a processing device, cause the processing device to carry out an operation (e.g., performing tumor fraction estimation, aligning sequencing data, and performing other sequencing analysis). In such an embodiment, the operation may be carried out on a singular device or between multiple devices (e.g., a server and a client device). A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 110, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G), 5^{th} (5G) generation, Long Term Evolution (LTE) radio access for cellular systems, WLAN, Wireless Router (WR) mesh, and the like. Access technologies such as 2G, 2.5G, 3G, 4G, 5G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present invention include apparatuses, systems, and methods implementing the Engine. Embodiments of the present invention may be implemented on one or more of client devices 102-106, which are communicatively coupled to servers including servers 107-109. Moreover, client devices 102-106 may be communicatively (wirelessly or wired) coupled to one another. In particular, software aspects of the Engine may be implemented in the program 223. The program 223 may be implemented on one or more client devices 102-106, one or more servers 107-109, and 113, or a combination of one or more client devices 102-106, and one or more servers 107-109 and 113.

A “DNA sample” refers to a nucleic acid sample derived from an organism, as may be extracted for instance from a solid tumor or a fluid. The organism may be a human, an animal, a plant, fungi, or a microorganism. The nucleic acids may be found in a solid sample such as a Formalin-Fixed Paraffin-Embedded (FFPE) sample. Further, nucleic acids may be found in a fresh-frozen sample. Alternately, the nucleic acids may be found in limited quantity or low concentration, in bodily fluids (e.g., blood or plasma). As circulating free DNA in bodily fluids. Circulating free DNA can include DNA shed from a tumor in a cancer patient (circulating tumor DNA).

A “nucleotide sequence” or a “polynucleotide sequence” refers to the sequence of nucleotides, such as cytosine (represented by the C letter in the sequence string), thymine (represented by the T letter in the sequence string), adenine (represented by the A letter in the sequence string), guanine (represented by the G letter in the sequence string) and uracil (represented by the U letter in the sequence string) in any polymer or oligomer of nucleotides. It may be DNA or RNA, or a combination thereof. It may be found permanently or temporarily in a single-stranded or a double-stranded shape. Unless otherwise indicated, nucleic acids sequences are written left to right in 5′ to 3′ orientation.

“Sequencing” refers to the process of determining the sequence of a polymer or oligomer of nucleotides.

“High Throughput Sequencing (HTS)” or “Next Generation Sequencing (NGS)” refers to real time sequencing of multiple sequences in parallel, typically between 50 and a few thousand base pairs per sequence. Exemplary NGS technologies include those from Illumina, Ion Torrent Systems, Oxford Nanopore Technologies, Complete Genomics, Pacific Biosciences, BGI, and others. Depending on the actual technology, NGS sequencing may require sample preparation with sequencing adaptors or primers to facilitate further sequencing steps, as well as amplification steps so that multiple instances of a single parent molecule are sequenced, for instance with PCR amplification prior to delivery to a flow cell in the case of sequencing by synthesis. HTS and NGS of a DNA library will produce a set of sequencing read data which can be processed by a bioinformatics computer in a bioinformatics workflow.

“Sequencing depth” or “sequencing coverage” or “depth of sequencing” refers to the number of times a genome has been sequenced. In targeted panels assay workflows, only a small subset of regions of interest in the whole genome is sequenced and it may therefore be reasonable to increase the sequencing depth without facing significant data storage and data processing overhead. Moreover, apart from the higher cost related to data storage and processing, the operational cost of an experimental NGS run, that is, loading a sequencer with samples for sequencing, also needs to be optimized by balancing the coverage depth and the number of samples which may be assayed in parallel in routine clinical workflows. Indeed, next generation sequencers are still limited in the total number of reads that they can produce in a single experiment (i.e., in a given run). The lower the coverage, the fewer reads per sample for the genomic analysis, and the higher the number of samples that can be multiplexed within a next generation sequencing run.

“Aligning” or “alignment” or “aligner” refers to mapping and aligning base-by-base, in a bioinformatics workflow, the sequencing reads to a reference genome sequence, depending on the application. For instance, in a targeted enrichment application where the sequencing reads are expected to map to a specific targeted genomic region in accordance with the hybrid capture probes used in the experimental amplification process, the alignment may be specifically searched relative to the corresponding sequence, defined by genomic coordinates such as the chromosome number, the start position and the end position in a reference genome. As known in bioinformatics practice, in some embodiments “alignment” methods as employed herein may also comprise certain pre-processing steps to facilitate the mapping of the sequencing reads and/or to remove irrelevant data from the reads, for instance by removing non-paired reads, and/or by trimming the adapter sequence as the end of the reads, and/or other read pre-processing filtering means. Exemplary bioinformatics data representations with different coordinate systems (absolute or relative position indexing, 0-based or 1-based, etc.) include the BED format, the GTF format, the GFF format, the SAM format, the BAM format, the VCF format, the BCF format, the Wiggle format, the GenomicRanges format, the BLAST format, the GenBank/EMBL Feature Table format, and other suitable formats.

“Coverage” or “sequence read coverage” or “read coverage” refers to the number of sequencing reads that have been aligned to a genomic position or to a set of genomic positions. In general, a genomic region with a higher coverage is associated with a higher reliability in downstream genomic characterization, in particular when calling variants.

“Variant calling” refers to the process of identifying, in a bioinformatics workflow, the sequence variants in the aligned reads relative to a reference sequence. In bioinformatics data processing, a variant is uniquely identified by its position along a chromosome (chr,pos) and its difference relative to a reference genome at this position (ref, alt). Variants may include single nucleotide permutations (SNPs) or other single nucleotide variants (SNVs), insertions or deletions (INDELs), copy number variants (CNVs), as well as large rearrangements, substitutions, duplications, translocations, and others. Preferably variant calling is robust enough to sort out the real sequence variants from variants introduced by the amplification and sequencing noise artifacts, for example. In a bioinformatics workflow, a variant caller may apply variant calling to produce one or more variant calls listed in any suitable format, such as Variant Calling File (VCF format). However, other file formats may be utilized. It is understood that ‘variant calling data’ is the result of variant calling analysis according to any known method of variant calling. In the described methods, variant calling data may be data obtained from sequencing of a cancer patient tumor sample. As non-limiting examples, the present method may use variant calling data obtained with a next generation sequencing targeted panel, whole exome sequencing assay, or whole genome sequencing assay.

“Tumor fraction” refers to the proportion or percentage of genetic material within a biological sample that originates specifically from tumor cells. This fraction is indicative of the presence and extent of tumor cells within the sample, typically assessed through various molecular and genetic analysis techniques.

### Genomic Analysis System

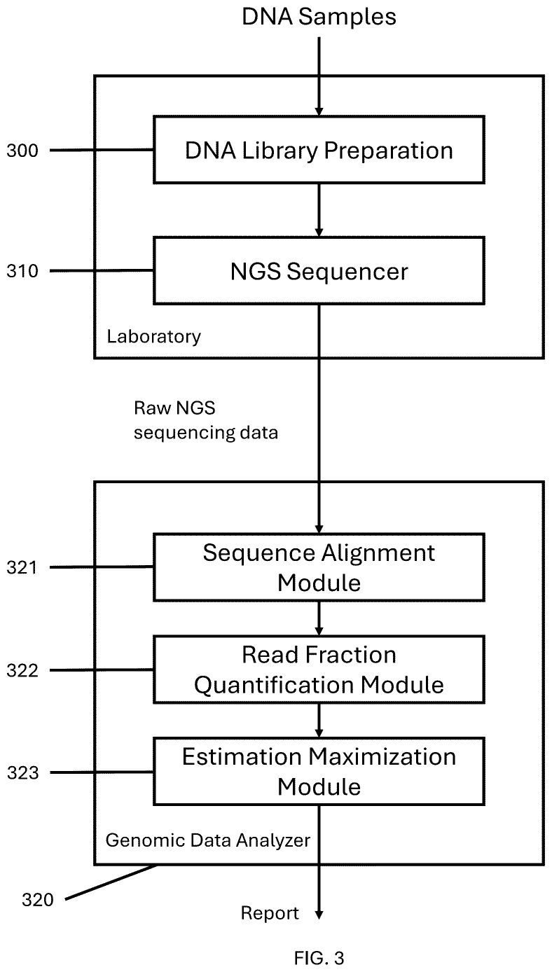

The proposed methods and systems will now be described by an exemplary genomic analysis system and workflow will now be described with further detail with reference to FIG. 3. As will be apparent to those skilled in the art of DNA analysis, a genomic analysis workflow comprises preliminary experimental steps to be conducted in a laboratory (also known as the “wet lab”) to produce DNA analysis data, such as raw sequencing reads in a next-generation sequencing workflow, as well as subsequent data processing steps to be conducted on the DNA analysis data to further identify information of interest to the end users, such as the detailed identification of DNA variants and related annotations, with a bioinformatics system (also known as the “dry lab”). Depending on the actual application, laboratory setup and bioinformatics platforms, various embodiments of a DNA analysis workflow are possible. FIG. 3 describes an example of an NGS system comprising a wet lab system wherein DNA samples are first experimentally prepared with a DNA library preparation protocol 300 which may produce, adapt for sequencing and amplify DNA fragments to facilitate the processing by an NGS sequencer 310. In a next generation sequencing workflow, the resulting DNA analysis data may be produced as a data file of raw sequencing reads in the FASTQ format. The workflow may then further comprise a dry lab Genomic Data Analyzer system 320 which takes as input the raw sequencing reads for a pool of DNA samples prepared according to the proposed methods and applies a series of data processing steps to characterize certain genomic features of the input samples. An exemplary Genomic Data Analyzer system 320 is the Sophia Data Driven Medicine platform (Sophia DDM) as already used by more than 1000 hospitals worldwide in 2024 to automatically identify and characterize genomic variants and report them to the end user, but other systems may be used as well. Different detailed possible embodiments of data processing steps as may be applied by the Genomic Data Analyzer system 320 for genomic variant analysis are described for instance in the international PCT patent application WO2017/220508, but other embodiments are also possible.

As illustrated on FIG. 3, the Genomic Data Analyzer 320 may comprise a sequence alignment module 321, which compares the raw NGS sequencing data to a reference genome, for instance the human genome in medical applications, or an animal genome in veterinary applications. In a conventional Genomic Data Analyzer system, the resulting alignment data may be further filtered and analyzed by a variant calling module (not represented) to retrieve variant information such as SNP and INDEL polymorphisms. The variant calling module may be configured to execute different variant calling algorithms. The resulting detected variant information may then be output by the Genomic Data Analyzer module 320 as a genomic variant report for further processing by the end user, for instance with a visualization tool, and/or by a further variant annotation processing module (not represented). In a possible embodiment, the Genomic Data Analyzer system 320 may comprise automated data processing modules such as a read fraction quantification module 322 to quantify read fractions at different tumor variants and representative positions, and an estimation maximization module 323 to estimate the tumor fraction based on iterative analysis, which may then be reported to the end user, for instance with a visualization tool, or to another downstream process (not represented).

### Data Processing Workflow

The Genomic Data Analyzer 320 may process the sequencing data to produce a genomic data analysis report by employing and combining different data processing methods.

The sequence alignment module 321 may be configured to execute different alignment algorithms. Standard raw data alignment algorithms such as Bowtie2 or BWA that have been optimized for fast processing of numerous genomic data sequencing reads may be used, but other embodiments are also possible. The alignment results may be represented as one or several files in BAM or SAM format, as known to those skilled in the bioinformatics art, but other formats may also be used, for instance compressed formats or formats optimized for order-preserving encryption, depending on the genomic data analyzer 320 requirements for storage optimization and/or genomic data privacy enforcement.

The Genomic Data Analyzer 320 may be a computer system or part of a computer system including a central processing unit (CPU, “processor” or “computer processor” herein), memory such as RAM and storage units such as a hard disk, and communication interfaces to communicate with other computer systems through a communication network, for instance the internet or a local network. Examples of genomic data analyzer computing systems, environments, and/or configurations include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, graphical processing units (GPU), and the like. In some embodiments, the computer system may comprise one or more computer servers, which are operational with numerous other general purpose or special purpose computing systems and may enable distributed computing, such as cloud computing, for instance in a genomic data farm. In some embodiments, the genomic data analyzer 320 may be integrated into a massively parallel system. In some embodiments, the genomic data analyzer 320 may be directly integrated into a next generation sequencing system.

The Genomic Data Analyzer 320 computer system may be adapted in the general context of computer system-executable instructions, such as program modules, being executed by a computer system. Generally, program modules may include routines, programs, objects, components, logic, data structures, and so on that perform particular tasks or implement particular abstract data types. As is well known to those skilled in the art of computer programming, program modules may use native operating system and/or file system functions, standalone applications; browser or application plugins, applets, etc.; commercial or open source libraries and/or library tools as may be programmed in Python, Biopython, C/C++, or other programming languages; and/or custom scripts, such as Perl or Bioperl scripts.

Instructions may be executed in distributed cloud computing environments where tasks are performed by remote processing devices that are linked through a communications network. In a distributed cloud-computing environment, program modules may be located in both local and remote computer system storage media including memory storage devices.

It is thus understood that methods described herein are computer-implemented methods. The NGS sequencer 310, the sequence alignment module 321, the read fraction quantification module 322, the estimation maximization module 323, and/or the genomic data analyzer 320, generally, may embody the electronic device 200 and/or components thereof. Accordingly, in one embodiment each of the modules depicted in FIG. 3 may be configured as standalone computer-executable instructions within the electronic device 200 and/or any one of the client devices 102-106 or servers 107-113. In an embodiment, one or more of the NGS sequencer 310, the sequence alignment module 321, the read fraction quantification module 322, the estimation maximization module 323, and/or the genomic data analyzer 320 may be configured as standalone hardware components. Improvements discussed herein to the tumor fraction determination workflow may facilitate improvements in both the adaptability of the method, but also the performance and efficacy of the hardware components executing the various steps of said method.

The solution as disclosed herein aims to determine the estimate tumor fraction (eTF) in cfDNA by identifying somatic variants from low pass whole genome sequencing data from the cfDNA sample of a cancer patient. The methods disclosed herein are contemplated in view of the aforementioned application (eTF in cfDNA for identifying somatic variants from lpWGS data); however, the versatility of the method and its steps extend beyond this scope, allowing for adaptation to various alternative use cases. While eTF in cfDNA is an exemplary embodiment, these methods demonstrate applicability across a spectrum of potential use cases, highlighting their flexibility and broad utility. Further, the methods described herein may be utilized in individuals with active cancer, those in remission, those who have recovered, and/or those seeking proactive monitoring.

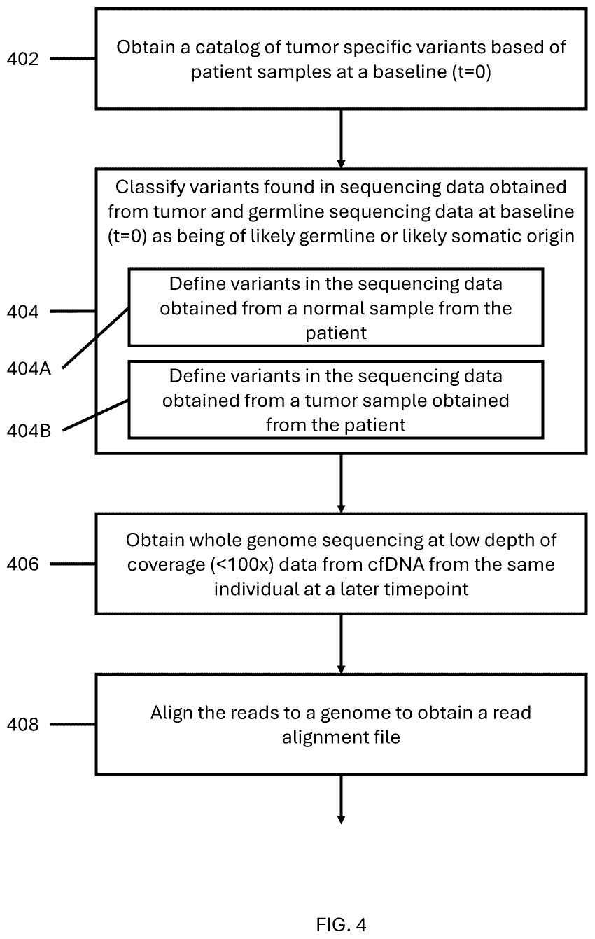

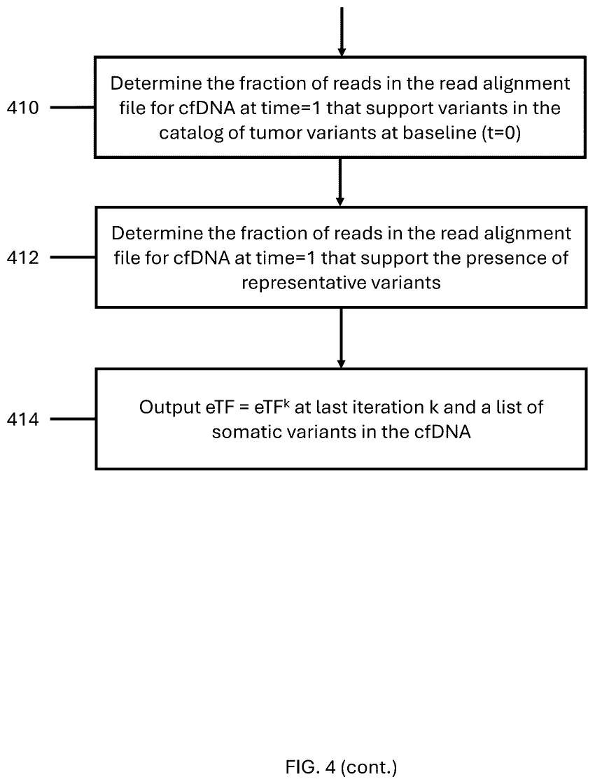

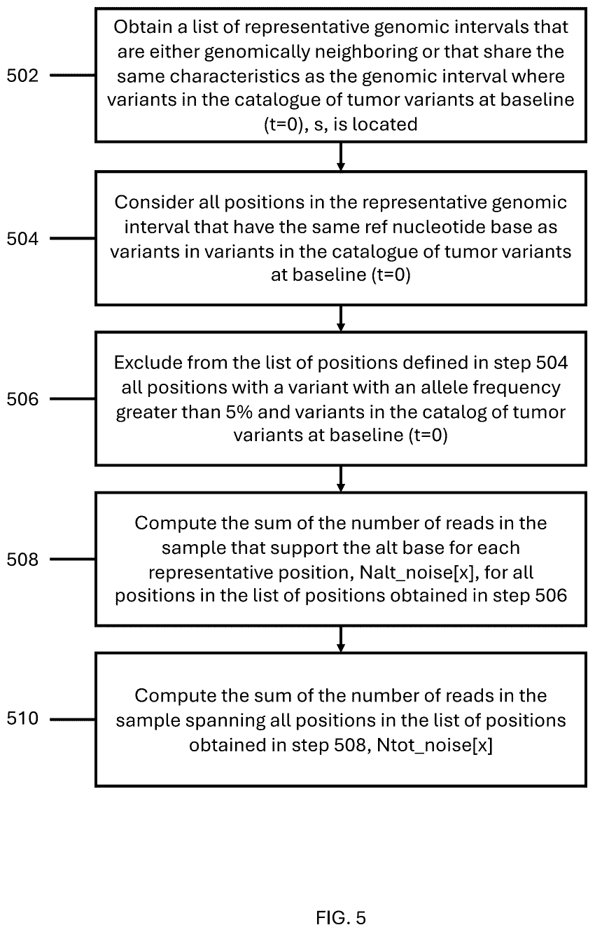

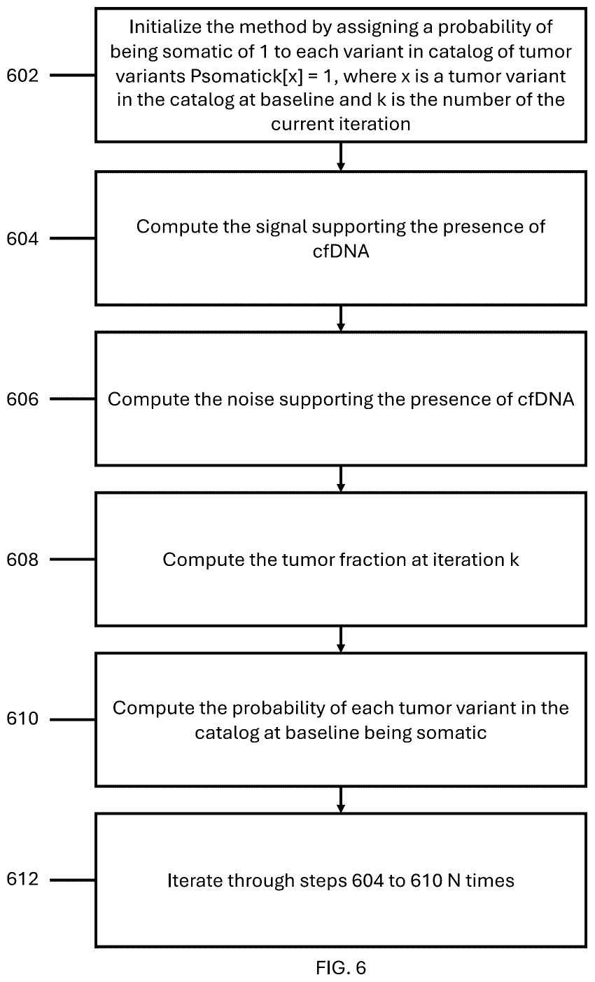

Accordingly, the method described herein is configured to distinguish true variants from sequencing noise using information solely present in the sequencing data from the patient. In an embodiment, the method may utilize tumor-normal pairs for listing tumor variants, and then only a cfDNA sample from the same patient to detect, for example, MRD. The proposed method may be based on estimating the tumor fraction in cfDNA based only on variant calling data obtained from whole genome sequencing data from tumor-normal pairs and cfDNA. The workflow for the solution is presented in at least FIGS. 4, 5, and 6.

Referring to FIG. 4, the computer-implemented method to estimate tumor fraction in circulating tumor DNA may comprise the following steps:

Step 402 may comprise obtaining a catalog of tumor specific variants based on or comprised of patient samples at a baseline (t=0). Tumor specific variants may be obtained from whole genome sequencing data from tumor and normal samples at baseline (t=0). Normal samples may either be blood or non-tumor tissue from the patient. For example, the normal sample may be derived from a patient's white blood cells. Tumor specific variants can alternatively be obtained from tumor only data.

Step 404 may comprise classifying variants found in sequencing data obtained from tumor and germline sequencing data at baseline (t=0) as being of likely germline or likely somatic origin.

Step 404A may comprise defining variants in the sequencing data obtained from a normal sample from the patient. In such an embodiment, variants found in this sample are determined to be likely of germline origin. Variants may be identified using standard variant calling approaches.

Step 404B may comprise defining variants in the sequencing data obtained from a tumor sample obtained from the patient. In such an embodiment, variants found in this sample are determined to be likely of germline origin if they are part of the list of variants found in step 404A, above. Yet further, variants not included in the list of germline variants and/or those that are not present in common variant databases are classified as being of likely somatic origin. In some embodiments, a common variant database may be referenced to determine whether variants are of likely somatic origin. However, the method herein may simply contemplate the list of germline variants when determining whether variants are of somatic origin, thus, obviating the need for a common variant database.

Step 406 may comprise obtaining whole genome sequencing at low depth of coverage (e.g., <100x) data from cfDNA from the same individual at a later timepoint. Although the method herein contemplates sequencing or obtaining sequencing data later, step 406 may be enacted in relative unison or in tandem with steps 402 and 404.

Step 408 may comprise aligning the reads to a genome to obtain a read alignment file (e.g., a BAM file).

Step 410 may comprise determining the fraction of reads in the read alignment file for cfDNA at time=1 that support variants in the catalog of tumor variants at baseline (t=0). However, in another embodiment, the determination of the fraction of reads in the read alignment file for cfDNA may occur in relative unison and/or in tandem with steps 402-408. For each variant in the catalog, at step 410, the method may comprise computing the number of reads in the sample that support the alternate (“alt”) sequence, (a sequence that provides an alternate representation of a locus relative to the reference sequence), Nalt_signal[x]; and computing the number of reads in the sample that span the variant, Ntot_signal [x].

Step 412 may comprise determining the fraction of reads in the read alignment file for cfDNA at time=1 that support the presence of representative variants. Although the determination of the fraction of reads in the read alignment file for cfDNA may occur at a time after steps 402-410, in another embodiment, such a determination may be made in relative unison and/or tandem with the execution of any of steps 402-410.

In an embodiment, step 412 further comprises representative variants identification via representative variants identification method 500.

Step 502 may comprise obtaining a list of representative genomic intervals that are either genomically neighboring or that share the same characteristics as the genomic interval where variants in the catalog of tumor variants at baseline (t=0), s, is located. In an embodiment, “genomically neighboring” may be defined as a window of 100 bp centered around variants in the catalog of tumor variants at baseline (t=0). However, such a window may be of any suitable size to capture variants as desired above. As a nonlimiting example, the window may be selected from lengths between 70 bp and 130 bp. In an embodiment, the “same characteristics” may be defined as similar characteristics including, for example, similar sequence composition or chromatin status. However, additional characteristics known to a person of ordinary skill in the art may be contemplated in assessing the list of representative genomic intervals.

Step 504 may comprise considering all positions in the representative genomic interval that have the same reference (“ref”) nucleotide base (a sequence that provides the same representation of a locus relative to the reference sequence), as variants in the catalog of tumor variants at baseline (t=0), (e.g., if variant x in a ref base is A, select all positions with a ref A from representative intervals).

Step 506 may comprise excluding from the list of positions defined in step 504 all positions with a variant with an allele frequency greater than 5% and variants in the catalog of tumor variants at baseline (t=0). Although, the method herein contemplates utilizing an allele frequency threshold of 5%, a person of ordinary skill in the art would recognize that other threshold percentages may be utilized within the spirit of step 506.

Step 508 may comprise computing the sum of the number of reads in the sample that support the alt base for each representative position, Nalt_noise[x], for all positions in the list of positions obtained in step 506.

Step 510 may comprise computing the sum of the number of reads in the sample spanning all positions in the list of positions obtained in step 508, Ntot_noise[x].

Step 414 may comprise using an iterative method 600 to estimate the fraction of ctDNA in the cfDNA sample (i.e., eTF) given the probability of variants observed in the cfDNA sample to be of somatic origin.

Step 602 may comprise initializing the iterative method 600 by assigning a probability of being somatic of 1 to each variant in the catalog of tumor variants, Psomatic^{k}[x]=1, where x is a tumor variant in the catalog at baseline and k is the number of the current iteration. Alternatively, the common variant database may be used to assign the probability of variants being somatic. In yet a further embodiment, the common variant database may be selectively configured for assigning the probability of a given variant being somatic, such that the method or administer of said method may utilize the common variant database for certain variants, while assigning a probability of 1 for other variants.

Step 604 may comprise computing the signal supporting the presence of each variant in the catalog of tumor specific variants in cfDNA using the weighted sum:
i. Nalt_signal=sum(Nalt_signal[x]*Psomatic^{k}[x]) over all x's
ii. Ntot_signal=sum(Ntot_signal[x]*Psomatic^{k}[x]) over all x's
iii. Signal=Nalt_signal/Ntot_signal

Step 606 may comprise computing the noise supporting the presence of each variant in the catalog of tumor specific variants in cfDNA using the weighted sum:
iv. Nalt_noise=sum(Nalt_noise[x]*Psomatic^{k}[x]) over all x's
v. Ntot_noise=sum(Ntot_noise[x]*Psomatic^{k}[x]) over all x's
vi. Noise=Nalt_noise/Ntot_noise

In an embodiment, Nalt_signal and Ntot_signal may be derived from the analysis of specific tumor variants, while Nalt_noise and Ntot_noise may be derived from the analysis of representative variants.

Step 608 may comprise computing the tumor fraction at iteration k as eTF^{k}=max(Signal−Noise, 0). In an embodiment, the max function may be configured to return the item with the highest value, or the item with the highest value in an iterable. In other embodiments, other functions known to a person of ordinary skill in the art may be utilized. The expectation-maximization algorithm contemplated above, may allow for estimation of parameters in statistical models (of particular utility in instances where data is incomplete or missing).

Step 610 may comprise computing the probability of each tumor variant in the catalog at baseline being somatic.

As a nonlimiting example, for each variant in the catalog, the likelihood of the variant being germline, Lgermline[x], may be computed as:

$$Lgermline [ x ] = Binomial ( Ntot\_signal [ x ] , 1 ; Nalt\_signal [ x ] ) + Binomial ( Ntot\_signal [ x ] , 0.5 ; Nalt\_signal [ x ] )$$

Further, as a nonlimiting example, for each variant, the likelihood of the variant being somatic may be computed as:

Lsomatic^{k}[x]=Binomial(Ntot_signal[x], eTF^{k}; Nalt_signal[x])

Yet further, as a nonlimiting example, the probability of variant x being somatic may be computed as:

Psomatic^{k}[x]=Lsomatic^{k}[x]/(Lsomatic^{k}[x]+Lgermline[x])

Although a binomial distribution is contemplated above, the likelihood of somatic and germline variants may be assessed via other probability distributions including, but not limited to, Poisson, negative binomial, and beta-binomial.

Step 612 may comprise iterating through steps 604 to 610 N times (e.g., N=3,5,10). Alternatively, the iteration may stop if the relative change (eTF^{k}−eTF^{k-1})/eTF^{k-1} is smaller than a predefined number; in effect, accounting for convergence or asymptotic behavior.

Step 414 may comprise generating and/or outputting eTF=eTF^{k} at last iteration k and a list of somatic variants in the cfDNA, wherein a variant is defined as being somatic if Psomatic^{k}[x] is greater than a predefined value p.

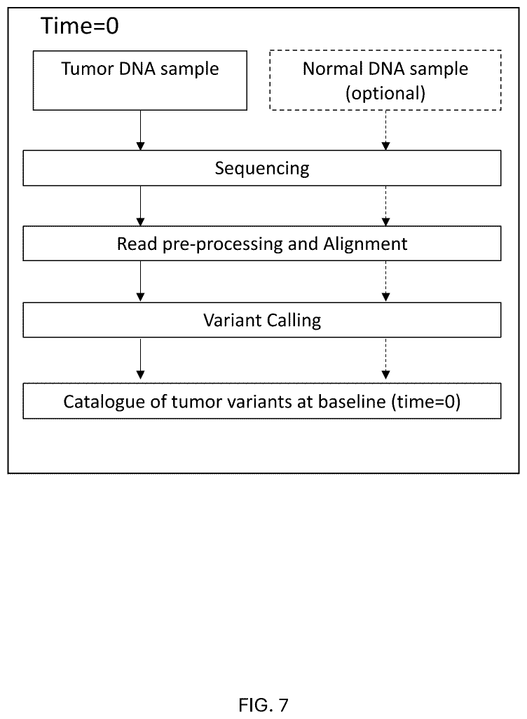

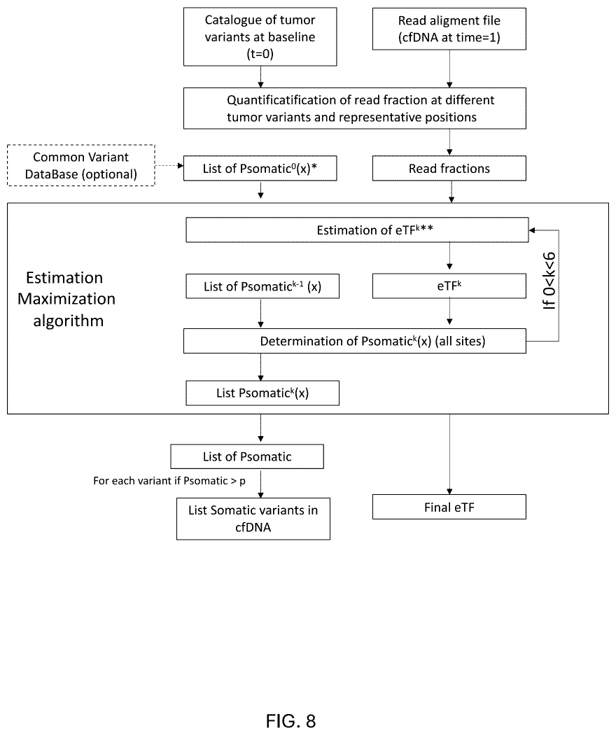

Referring to FIGS. 7-8, the list of Psomatic may be the list of probability of being somatic at all sites, accordingly, the initial Psomatic for a variant x in iteration 0. The eTF(k) may be the eTF at iteration k, where k=[1-5].

The method described herein provides for probabilistic determination of tumor fraction and somatic variants, offering the advantage of quantifying uncertainties and providing a statistical framework for predictions, which enhances the reliability of the results. Accordingly, the method above, and specifically step 414 and iterative method 600, permits the integration of diverse data sources, providing a source-agnostic analysis. Unlike heuristic determination, which relies on an expert's assessment or subjective threshold analysis, probabilistic methods are reproducible, consistent, and universal. Thus, traditional methods of tumor fraction analysis utilizing conventional cancer type-specific metrics have limited applicability across tumor profiles, differing patients, and cancer types.

The method described herein is configured to minimize the impact of false positives. As a nonlimiting example, if there is a germline variant within the list of tumor specific variants, it would follow that such a germline variant was inadvertently missed during the initial diagnostic. Accordingly, especially in low coverage embodiments, such germline variant false positives will manifest a very high signal in, for example, the cfDNA. The method described above is, thus, configured to detect such inadvertent germline variants and remove said variants from the analysis, providing a more accurate and representative tumor fraction determination.

The method described herein may be utilized in conjunction with any workflow including direct or indirect tumor fraction estimation and a list of tumor specific variants exists. Thus, the workflow presented herein is adaptable to various data sources, wet lab setups, and dry lab configurations, permitting the workflow to be input-agnostic. An exemplary use case includes using the tumor fraction estimation in MRD analysis. For example, presence of MRD may be calculated or determined by assessing the estimated tumor fraction and, if larger than a threshold value, the sample may be marked as MRD positive, and a treatment recommendation may be triggered for the patient.

Although whole genome sequencing is contemplated herein, other sequencing methods may be utilized such as whole exome sequencing or large gene panels. However, in instances where large gene panels are utilized, that large gene panels should be of sufficient size to encompass the variety and quantity of potential variants.

Referring to FIG. 7, a catalog of tumor variants may be generated at a baseline. In such an embodiment, at least a tumor DNA sample may be sequenced to generate reads, which may be preprocessed and aligned. After such preprocessing and alignment, standard variant calling methods may be employed to generate the catalog of tumor variants at baseline. In a further embodiment, a normal DNA sample may be sequenced in conjunction with the sequencing of the tumor DNA sample.

Referring to FIG. 8, the catalog of tumor variants at baseline may be assessed by the read fraction quantification module 322 to quantify read fractions at different tumor variants and representative positions. In a further embodiment, the read alignment file, for example, of cfDNA taken separately than the baseline, may be passed through the read fraction quantification module 322 to output read fractions. Optionally, a list containing the probability of a variant being somatic may be passed from a common variable database to the estimation maximization module 323 to estimate the tumor fraction based on iterative analysis.

In a possible embodiment, the read fraction quantification module 322 and/or the estimation maximization module 323 are computer-implemented algorithms trained to quantify read fractions at different tumor variants and representative positions, and to estimate the tumor fraction based on iterative analysis, respectively.

In a possible embodiment, in the case of a tumor sample, the estimation maximization module 323 may identify a tumor fraction that is indicative of a particular presence (e.g., MRD, ctDNA, etc.) and may thus require a given treatment.

The estimation maximization module 323 or the genomic data analyzer 320, more generally, may accordingly identify, categorize and report the estimated tumor fraction. In a preferred embodiment, the estimation maximization module 323 or the genomic data analyzer 320 may accordingly identify, categorize and report a tumor fraction and its corresponding indication, such as MRD-negative, MRD-positive, ctDNA-negative, ctDNA-positive, the presence of particular variants, and the like. In a possible embodiment, the estimation maximization module 323 or the genomic data analyzer 320 may report the list of somatic variants in conjunction with the tumor fraction for a given sample. As will be apparent to those skilled in the art, this bioinformatics method then significantly facilitates detailed understanding of the underlying sample and/or patient and the recourse for treatment based on the identified somatic variants and tumor fraction.

In one embodiment, the somatic variant and tumor fraction report may be displayed to the end user on a display or a graphical user interface. Further, after generation of the list of somatic variants and/or tumor fraction, such a report may be converted to any usable file, allowing automated processing and/or utilization by another program.

The disclosure provides a method providing a report on tumor fraction and/or list of somatic variants, which may be utilized to guide and/or optimize treatment strategies for cancer patients based on the findings in the report. In an embodiment, the report and/or data thereof may be integrated into preexisting treatment protocols or health management systems, ensuring that healthcare providers, clinicians, and/or other related facilities have timely access to critical information. Therefore, by incorporating the list of somatic variants and/or the tumor fraction data into the decision-making process, the system enhances the precision and effectiveness of personalized treatment plans. Such an effect may be especially apparent in instances where input data is limited to the types contemplated herein (tumor-normal pairs, cfDNA via white blood cell samples, etc.). The methods described herein, specifically the estimation of tumor fraction via iterative means, reduces the potential for human error and improves the overall workflow by distinguishing true variants from sequencing noise using information solely present in the sequencing data from the patient.

Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

Various elements, which are described herein in the context of one or more embodiments, may be provided separately or in any suitable subcombination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claims.

All references, patents and patent applications and publications that are cited or referred to in this application are incorporated in their entirety herein by reference. Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A computer-implemented method for estimating a tumor fraction in a patient sample, comprising the steps of:
obtaining a catalog of tumor specific variants,
wherein the catalog of tumor specific variants was assembled based on analysis of at least one patient sample at a baseline (t=0);
obtaining whole genome sequencing data from the patient sample;
aligning reads of the whole genome sequencing data from the patient sample to a reference genome to obtain a patient sample read alignment file;
determining a measure of a signal supporting a presence, in the patient sample read alignment file, of variants in the catalog of tumor specific variants;
determining a measure of noise associated with variants similar to variants in the catalog of tumor specific variants in the patient sample read alignment file;
estimating, over one or more iterations, k, the fraction of tumor (eTF) DNA in the patient sample given the measure of the signal and the measure of the noise at all tumor specific positions; and
generating a final eTF at a last iteration of the one or more iterations and a list of somatic variants in the patient sample.

2. The computer-implemented method of claim 1, the step of estimating the eTF further comprising the steps of:
assigning a probability of being somatic to each variant in the catalog of tumor specific variants;
computing a signal supporting the presence of the variants in the catalog of tumor specific variants in the patient sample using a first weighted sum; and
computing a noise supporting the presence of the variants in the catalog of tumor specific variants in the patient sample using a second weighted sum.

3. The computer-implemented method of claim 2, wherein, at k=0, the probability of being somatic of each variant in the catalog of tumor specific variants is 1, Psomatic^{k}[x]=1, wherein x is a tumor variant in the catalog of tumor specific variants and k is the number of a current iteration.

4. The computer-implemented method of claim 2, wherein the probability of being somatic to each variant in the catalog of tumor specific variants, Psomatic^{k}[x], at k=0, is based on information for the variant obtained from a common variant database.

5. The computer-implemented method of claim 2, the step of computing a probability of each tumor variant in the catalog of tumor specific variants being somatic, Psomatic^{k}[x], further comprising the steps of:
computing, at k>0, for each variant in the catalog of tumor specific variants, a likelihood of the variant being germline (Lgermline[x]),
wherein Lgermline[x] is Binomial(Ntot_signal[x], 1; Nalt_signal[x])+Binomial(Ntot_signal[x], 0.5; Nalt_signal[x]);
computing, at k>0, for each variant in the catalog of tumor specific variants, a likelihood of the variant being somatic (Lsomatic^{k}[x]),
wherein Lsomatic^{k} [x]=Binomial(Ntot_signal[x], TF^{k}; Nalt_signal[x]); and
computing, at k>0, the probability of variant x being somatic,
wherein Psomatic^{k}[x]=Lsomatic^{k} [x]/(Lsomatic^{k} [x]+Lgermline[x]).

6. The computer-implemented method of claim 5, the step of determining a fraction of reads in the read alignment file supporting presence of representative variants further comprising the steps of:
obtaining a list of representative genomic intervals;
considering positions in each of the representative genomic intervals in the list of representative genomic intervals that have a same reference nucleotide base (ref nucleotide base) as variants in the catalog of tumor specific variants;
excluding, from the positions in each of the list of representative genomic intervals having the same reference nucleotide base as variants in the catalog of tumor specific variants, and excluding positions comprising a variant with an allele frequency greater than 5% and variants in the catalog of tumor specific variants.

7. The computer-implemented method of claim 6, wherein, in the list of representative genomic intervals, each of the representative genomic intervals are genomically neighboring, defined as a window of 100 bp centered around variants in the catalog of tumor specific variants.

8. The computer-implemented method of claim 6, wherein, in the list of representative genomic intervals, each of the representative genomic intervals share one or more similar characteristics as the genomic interval where variants in the catalog of tumor specific variants are located.

9. The computer-implemented method of claim 8, wherein the similar characteristics include similar sequence composition and chromatin status.

10. The computer-implemented method of claim 5, wherein the measure of the signal supporting the presence of the variants in the catalog of tumor specific variants in the patient sample using a first weighted sum is defined as Signal=Nalt_signal/Ntot_signal,
wherein Nalt_signal=sum(Nalt_signal[x]*Psomatic^{k}[x]) over all variants, x, and
wherein Ntot_signal=sum(Ntot_signal[x]*Psomatic^{k}[x]) over all variants, x.

11. The computer-implemented method of claim 10, further comprising the steps of:
for each variant in the catalog of tumor specific variants, computing a number of reads in the patient sample that supports an alt sequence (Nalt_signal[x]); and
for each variant in the catalog of tumor specific variants, computing a number of reads in the patient sample that span the variant (Ntot_signal[x]).

12. The computer-implemented method of claim 5, wherein the measure of the noise supporting the presence of the variants in the catalog of tumor specific variants in the patient sample using a second weighted sum is defined as Noise=Nalt_noise/Ntot_noise,
wherein Nalt_noise=sum(Nalt_noise [x]*Psomatic^{k}[x]) over all variants, x,
wherein Ntot_noise=sum(Ntot_noise [x]*Psomatic^{k}[x]) over all variants, x.

13. The computer-implemented method of claim 12, further comprising the steps of:
determining a fraction of reads in the patient sample read alignment file supporting representative variants;
computing a number of reads in the patient samples that supports an alt sequence (Nalt_noise[x]) at representative variant; and
computing a number of reads in the patient samples that span the variant (Ntot_noise [x]) at representative variant.

14. The computer-implemented method of claim 13, wherein whole genome sequencing data from the patient sample has a coverage less than 5x.

15. The computer-implemented method of claim 1, wherein the catalog of tumor specific variants is obtained from whole genome sequencing data from tumor-normal pairs.

16. The computer-implemented method of claim 14, further comprising the steps of:
determining, via variant calling methods, variants present in the tumor sequencing data at the baseline and the germline sequencing data at the baseline;
classifying the variants present in the tumor sequencing data at the baseline and the germline sequencing data at the baseline as likely germline or likely somatic; and
comparing the likely germline variants and the likely somatic variants to construct the catalog of tumor specific variants.

17. The computer-implemented method of claim 16, the step of classifying the variants present in the tumor sequencing data at the baseline and the germline sequencing data at the baseline as likely germline or likely somatic further comprising the step of:
defining variants in the germline sequencing data at the baseline as likely germline.

18. The computer-implemented method of claim 17, the step of classifying the variants present in the tumor sequencing data at the baseline and the germline sequencing data at the baseline as likely germline or likely somatic further comprising the step of:
defining variants in the tumor sequencing data at the baseline as likely germline if said variants are present in the variants of the germline sequencing data at the baseline.

19. The computer-implemented method of claim 18, the step of classifying the variants present in the tumor sequencing data at the baseline and the germline sequencing data at the baseline as likely germline or likely somatic further comprising the step of:
defining variants not included in the germline sequencing data at the baseline as likely somatic.

20. The computer-implemented method of claim 19, wherein the patient samples comprise cell-free DNA (cfDNA), wherein the estimating, over one or more iterations, k, the fraction (eTF) of tumor DNA in the patient sample given the measure of the signal and the measure of the noise at all tumor specific positions further comprises utilizing an expectation-maximization algorithm, wherein a variant is defined as being somatic if Psomatic^{k}[x] is greater than a predefined value, p, wherein the tumor DNA is circulating tumor DNA (ctDNA).
